(19) United States
(12) Patent Application Publication
(10) Pub. No.: US 2014/0378658 A1
(43) Pub. Date: Dec. 25, 2014

(54) NEUROLOGICAL THERAPIES

(71) Applicants: Instituto Superiore di Sanita, Roma (IT); Alma Mater Studiorum - Universita di Bologna, Bologna (IT)

(72) Inventors: Carla Fiorentini, Roma (IT); Stefano Loizzo, Roma (IT); Fiorella Malchiodi-Albedi, Roma (IT); Sara Travaglione, Roma (IT); Roberto Rimondini, Bologna (IT); Gabriele Campana, Bologna (IT)

(73) Assignee: Alma Mater Studiorum-Universita di Bologna, Bologna (IT)

(21) Appl. No.: 14/372,913

(22) PCT Filed: Jan. 21, 2013
(86) PCT No.: PCT/EP2013/051061

§ 371 Date: Jul. 17, 2014

(30) Foreign Application Priority Data
Jan. 20, 2012 (GB) 1201138.3

(51) Int. Cl. A61K 38/16

(52) U.S. Cl. CPC A61K 38/164
USPC 530/350

(57) ABSTRACT

Cytotoxic necrotising factor 1 (CNF1) treats dysfunctional astrocytes and is useful to treat conditions associated with astrogliosis and/or neuroinflammation.

The present invention relates to the use of CNF1 in the treatment, prevention, and prophylaxis of neurological disorders, and to medicaments therefor.

Proteins belonging to the Rho GTPases' family act as molecular switches that cycle between a GDP-bound inactive and a GTP-bound active state to transduce extracellular signals to the actin cytoskeleton. Their ability to modulate the organisation of the actin network plays an important role in the morphogenesis of the dendritic spines of neurons in the brain and synaptic plasticity (Hotulainen and Hoogenraad 2010). In the nervous system, the Rho GTPases play a key role in several processes, and mutations in proteins involved in Rho GTPase signaling may be causative in some forms of mental retardation.

We have previously shown that a bacterial protein toxin from E. coli, cytotoxic necrotising factor 1 (CNF1), which acts by permanently activating the Rho GTPases (Flatau et al., 1997; Schmidt et al., 1997), can influence neuronal plasticity in the central nervous system (CNS). CNF1 is a 113.8 kDa protein, produced by several Escherichia coli strains, and we have demonstrated that CNF1, by directly modulating the brain Rho GTPases, can i) enhance cognitive performances in wild-type mice (C57BL/6J) (Diana et al., 2007; De Viti et al., 2010), ii) counteract the formalin-induced inflammatory pain in mice, after both peripheral and central administration (Pavone et al., 2009), and iii) trigger structural remodelling and functional plasticity into the adult rat visual cortex (Cerri et al., 2011).

Surprisingly, we have now found that CNF1 does not affect the neurons directly, but acts via the astrocytes. The advantage is that this is of assistance in the treatment of neuroinflammatory conditions, such as multiple sclerosis, neuropathic pain, etc. and in the treatment of astrogliosis.

Thus, in a first aspect, the present invention provides CNF1 for use in the treatment of a condition associated with astrocyte dysfunction. The condition to be treated excludes conditions known to be treatable in the art as described above.

The term ‘treatment’ includes prevention, inhibition, and prophylaxis, unless otherwise apparent from the context.

Conditions to be treated are preferably those associated with neuroinflammation and/or astrogliosis, but as demonstrated herein, CNF1 effectively restores dysfunctional astrocytes to normal functioning, and may be used wherever abnormal astrocyte function is implicated.

CNF1 was originally isolated from E. coli, and a preferred sequence is disclosed herein as SEQ ID NO. 1. It will be appreciated that it is not critical to use exactly the polypeptide of SEQ ID NO 1, and nor is it necessary to use 100% of the sequence. It is only necessary that the peptide used retains the ability to activate the Rho GTPases. Otherwise, it may be a mutant or variant thereof and may be modified by deletion, insertion, or inversion of short sequences, such as up to 10 amino acids in length, preferably no more than 5 amino acids. Separately, or in addition, amino acids may be substituted by other amino acids, preferably those which will not severely disrupt any tertiary structure, such as alpha or beta structure, or binding sites.

The CNF1 may also be bound to a carrier molecule for expression and/or formulation purposes, for example.

CNF1 may be administered to any suitable patient. Patients are preferably mammals, and preferably human. The condition to be treated may be manifest, or may be incipient, such as in the case of neurological trauma, where use is preventative if administered straight away, for example.

CNF1 may be administered in any suitable form. As CNF1 affects the astrocytes, it will often be necessary to administer it by injection or drip, so that the formulation will be liquid suitable for injection, and may incorporate buffering, isotonic agents, and/or preservatives, for example.

Other suitable forms may be creams, ointments, gels, drops, unguents, pessaries, suppositories, transdermal patches, and any form indicated by a skilled physician.

CNF1 may be used in any concentration deemed suitable by a skilled physician, but may be used in concentrations as low as 10⁻¹¹ M or up to 10⁻⁶ M or higher, depending on the age, sex, health, or other pertinent parameter, of the patient.

We have found that CNF1 acts specifically on astrocytes, the most abundant type of glial cells in the central nervous system (CNS), which are involved in the induction of neuroinflammation, and not on neurons, as previously thought, thereby providing treatments not apparent to one skilled in the art prior to this finding.

To define the mechanisms by which CNF1 can ameliorate the neuronal function, we analysed the effects of the toxin on primary neuronal and astrocytic cultures. We discovered that the cellular targets of CNF1 are not directly neurons, but astrocytes that, under the influence of CNF1, increase their supporting activity on neuronal growth and differentiation. Astrocytes, the most abundant type of glial cells in CNS, are involved in the neuroinflammatory response that characterises many CNS diseases, including cerebrovascular disease, seizure disorders/epilepsy, neuropathic pain, Parkinson's and Alzheimer's diseases (Sofroniew and Vinters, 2010).

Under stress and injury, astrocytes become astrogliotic leading to an upregulation of glial fibrillary acid protein (GFAP), proinflammatory cytokines and chemokine release, hypertrophy and a decrement of purines such as ATP. In particular, we observed that the production of interleukin 1β (IL-1β), known to reduce dendrite development and complexity in neuronal cultures (Gilmore J H, et al., 2004), was decreased in CNF1-exposed astrocytes.

The beneficial role of CNF1 was confirmed in transgenic mice homozygous for human ApoE4 (APOE4 TR). The apolipoproteins (APO) are cholesterol transporters of high importance for neuronal plasticity, glucose utilisation and mitochondrial functions and the gene ApoE4 has been indicated as a risk factor in diseases with a dysregulation of lipoprotein metabolism and transport, and alterations in immune regulation, such as atherosclerosis and familial dysbetalipoproteinemia, Alzheimer's disease (Verghese et al., 2011). In ApoE4 mice, treatment with CNF1 reduced astrogliosis, IL-β and β amyloid expressions, and increased ATP levels, all of which may ameliorate neuronal functionality. None of these responses were observed in the non-symptomatic control variant (APOE3 mice) challenged with CNF1.

Furthermore, neuronal and astroglial dysfunction and inflammatory changes, such as increased GFAP-immunoreactivity and proinflammatory cytokine levels (Gahring et al., 1997; Li et al., 2007; Pernot et al., 2011) also characterise spontaneous seizure (Tan et al., 2008; Bortolato et al., 2010; Reid et al., 2011). We therefore performed electroencephalography studies (EEG) to evaluate frequency and time domain in the somatosensitive cortex of a genetic spontaneous seizures model, the DBA/2J (D2) mouse. D2 presents low frequency (7-8 cycles per second) spike and wave complexes, events significantly blocked by i.c.v. injection of CNF1.

Thus, we have shown, for the first time, that CNF1 can act specifically on glial cells by reducing the production of the pro-inflammatory cytokine IL-1β in vitro and in vivo. In addition, astrocytes challenged with the toxin are able to provide a more efficient substrate to neuronal growth in primary neuronal cultures. We have now established that it is astrocytes and not neurons that are pivotal in the enhanced neurotransmission and synaptic plasticity previously observed after in vivo treatment with CNF1 (Diana et al., 2007; Cerri et al., 2011).

The uses and advantages of CNF1 include:

1. CNF1 is capable of inducing a decrease in GFAP expression. GFAP is the main intermediate filament protein in mature astrocytes, but also an important component of the cytoskeleton in astrocytes during development. GFAP has been shown to be involved in astrocyte functions relevant to CNS regeneration and synaptic plasticity. Several lines of evidence suggest that the observed reduction in GFAP content in CNF1-treated astrocytes could be related to the increased dendritogenesis. GFAP has in fact been found to be a negative regulator of astrocytic ability to improve neuronal growth and neuritogenesis (Menet et al. 2001). In addition, highly reactive astrocytes, as shown by GFAP immunostaining, induce the formation of fewer synaptic contacts in co-cultured neurons, compared to less reactive astrocytes (Emirandetti et al., 2006). Recent studies have shown that increased astrocytic GFAP expression can be related to neuron atrophy, whereas diminished GFAP content restores neurite outgrowth in certain conditions (Rozovsky et al., 2005). It is noteworthy that various pathologic conditions of the CNS are accompanied by reactive gliosis, which is characterised by an increase in the expression of GFAP and is considered to have a role in neurodegeneration (Middeldorp and Hol, 2011). Thus, the capacity of CNF1 to modulate GFAP content provides a use in the treatment of those neurological diseases where astrocytosis contributes to neuronal damage.

2. It is known that the secretion of pro-inflammatory cytokines is up-regulated in GFAP-overexpressing, activated astrocytes, and it is believed to contribute to neurodegeneration (Whitney N P, et al., 2009). In our models, both in vitro and in vivo, when exposed to CNF1, astrocytes reduced the secretion of IL-1β. These results are in line with previous reports stating that IL-1β can significantly reduce dendrite development and complexity in neuronal cultures (Gilmore J H, et al., 2004). In addition, upregulation of IL-1β was observed to negatively influence neurogenesis (Kuzumaki N, et al., 2010) and neurodevelopment (Garay P A, et al., 2010), possibly by interfering with the signalling of BDNF, a major trophic factor in the CNS, and critical for the development and survival of certain neuronal populations (Tong L, et al., 2008). IL-1β is considered to contribute to neurotoxicity in several CNS diseases, and CNF1 is useful in the treatment of those conditions where upregulation of proinflammatory cytokines is of pathogenic relevance.

3. Astrocytes are an important source of ATP release in the CNS and have a number of mechanisms for the release of ATP. There is compelling evidence that astroglial ATP regulates neuronal synaptic strength, although the physiological significance of this astrocyte-to-neuron signalling is not certain. CNF1 is useful to rescue low ATP levels in the hippocampus of ApoE4 mice to normal levels.

4. CNF1 is useful to counteract central neuroinflammation. Neuroinflammation has been indicated in all diseases associated with reactive astroglyosis and its consequence (Glass et al., 2010), such as alterations in immune regulation, GFAP up-regulation and hypertrophy and a decrement of purines such as ATP. Examples are seizure disorders/epilepsy, glaucoma, Parkinson's disease, cerebral amyloid angiopathy and tauopathies (Sofroniew and Vinters, 2010).

Conditions treatable by use of CNF1, and associated with neuroinflammation and/or astrogliosis treatable by exposure of astrocytes to CNF1, include:

### Diabetic Retinopathy

In diabetic retinopathy (DR), activation of Muller glia, a subset of astroglial cells, has been found to contribute to the development of the disease. The present invention provides CNF1 for use in the treatment of DR. Typically, this might be achieved by the administration of either as eye drops or as intravitreal injection, both of which are routinely used in humans for delivering therapeutic agents in the posterior segment of the eye, such as, for example, anti-VEGF antibodies in Age-related Macular Degeneration.

### Glaucoma

Glaucoma is characterised by unexplained loss of retinal ganglion neurons and by the reactive gliosis of astrocytes and related Muller cells in the retina, and the reactivity of astrocytes that surround ganglion cell axons in the optic nerve head. CNF1 may be administered as intravitreal injection, a procedure used in the clinic for glaucoma treatment.

### Seizure Disorders/Epilepsy

Reactive astrogliosis is variable, but often prominent, in almost all forms of seizures. To treat seizures in hippocampal and cortical areas, an intraventricular injection of CNF1 is indicated.

### Parkinson's Disease

The molecular mechanisms underlying the pathogenesis of idiopathic Parkinson's disease (PD) involve the degeneration of the nigrostriatal system, and neuroinflammation plays a central role. CNF1 may be administered by bilateral striatal infusion is the procedure indicated for a pharmacological therapy for Parkinson's disease patients.

### Cerebral Amyloid Angiopathy

CAA (Cerebral amyloid angiopathy) refers to the deposition of β-amyloid, mainly amyloid β-40, in the media and adventitia of arterioles of the leptomeninges, causing, even in the absence of dementia, inflammation, vascular oedema and uncontrolled influx of peripheral blood components into the brain parenchyma and reactive astrogliosis. This is normally a condition found in the elderly. ApoE4 alleles are associated with increased risk of CAA, with apoE4 occurring in the perivascular space and in perivascular astrocytes around the vessels. CNF1 may be administered as intraventricular injections for this condition, for example.

### Alzheimer's Disease

Reactive astrogliosis is a well-known feature of Alzheimer's disease (AD). Reactive astrogliosis tends to be focal in AD such that reactive astrocytes are intimately associated with amyloid plaques or diffuse deposits of amyloid and surround them with dense layers of processes as if forming miniature scars around them, perhaps to wall them off and act as neuroprotective barriers. Reactive astrocytes can contain substantial amounts of different forms of amyloid beta, including amyloid beta 1-42 (Aβ42) as well as truncated forms. Reactive astrocytes can take up and degrade extracellular deposits of Aβ42 and that this function is attenuated in ApoE−/− astrocytes, suggesting that reactive astrocytes functions or dysfunctions could play a role in the progression and severity of AD. The intensity of reactive astrogliosis, as determined by GFAP levels, has been reported to increase in parallel with increasing progression of Braak stages in AD, while concomitantly the levels of astrocyte glutamate transporters have been reported to decline, thereby increasing the vulnerability of local neurons to excitotoxicity. CNF1 may usefully be administered as an intraventricular injection, for example.

### BRIEF DESCRIPTION OF THE DRAWINGS

The invention is further illustrated by the following Examples, in which the Figures are as follows:

FIG. 1 shows how CNF1 modifies neuritic tree and synapse development in neurons during differentiation;

FIG. 2 shows how CNF1-treated astrocytes provide a more efficient substrate to neuritogenesis and synaptogenesis;

FIG. 3 shows how CNF1 treatment endows astrocytes with a neuroprotective phenotype;

FIG. 4 shows how astrogliosis and neuroinflammation in ApoE4 mice is reversed by CNF1;

FIG. 5 shows how CNF1 increases the ATP levels and decrease the β amyloid expression in ApoE4 mice;

FIG. 6 shows how CNF1 counteracts spontaneous seizure in DBA/2J (D2) mice;

FIG. 7 illustrates astrocyte stellation after exposure to CNF1;

FIG. 8 shows spectrograms computed from EEG of absent seizures model (DBA/2J) treated with CNF1; and

FIG. 9 shows the distribution of scores in percent of total for control and CNF1 treated animals.

The following Examples are for illustrative purposes only, and are not intended to restrict the present invention in any way.

### EXPERIMENTAL

### Example 1

### Materials and Methods

### Primary Cultures

All primary cultures were obtained from Wistar rat embryos at gestational day 18 (Charles River). This study was carried out in strict accordance with the recommendations in the Guide for the Care and Use of Laboratory Animals of the National Institutes of Health. The protocol was approved by the Committee on the Ethics of Animal Experiments of the University of Minnesota (Permit Number: 27-2956). After dissection, hippocampus were dissociated in trypsin and plated on poly-L-lysine-coated glass coverslips in Minimum Essential Medium (MEM), containing 10% fetal calf serum; after two hours, the medium was replaced with Neurobasal Medium (NBM) supplemented with B27. To obtain pure neuronal cultures, hippocampal neurons were treated at day-in-vitro (DIV) 1 with 1.5 mM Arabinosyl-Cytosine (Ara-C). In these conditions, neuronal cultures contain 1-2% of Glial Fibrillary Acidic Protein-positive astrocytes (Malchiodi-Albedi et al, 2001). Primary astrocytic cultures were obtained from the cortex of rat embryos. After dissection and dissociation, as already described, cortical cell suspension was seeded in flasks in MEM, containing 10% fetal calf serum and allowed to grow to confluence. Cells were replated twice to obtain a cell culture highly enriched in astrocytes. Contamination of microglial cells was below 1%, as shown by staining with Bandeiraea simplicifolia lectin-peroxidase conjugate (data not shown). For primary astrocytic-neuronal co-cultures, astrocytes were first seeded on glass coverslips and allowed to grow to confluence. Hippocampal neuron suspension, obtained as described above, was seeded on the astrocytic monolayer and treated at DIV 1 with Ara-C, to block further growth of astrocytes. All cell cultures were grown at 37° C. in 5% CO₂.

### CNF1 Preparation and Treatments

CNF1 was obtained from the 392 ISS strain (provided by V. Falbo, Rome, Italy) and purified essentially as previously described (Falzano et al., 1993) with a few modifications in the procedure. For all experiments, a concentration of 10⁻¹⁰ M CNF1 was used.

### In Cultured Cells:

CNF1 was administered to pure neuronal cultures at day-in-vitro (DIV) 2 until fixation (5 or 9 or 14 DIV). Confluent primary astrocytic cell cultures were treated for 48 h with CNF1, after which the CNF1-containing medium was changed with CNF1-free NBM-B27 and primary hippocampal neurons were seeded on the astrocytic monolayer. In control cultures, hippocampal neurons were seeded on untreated astrocytes. Neuronal-astrocytic co-cultures were fixed at DIV 14.

### In Mouse Brains:

Animal surgery—After general anesthesia (2% Fluoxethane, air flow 1.8 l/min, Ugo Basile gas anesthesia), a needle connected to a 10-ml Hamilton microsyringe was placed in the lateral ventricle of the right cerebral hemisphere with a stereotactic technique (AP 0.1 mm, L ±0.9 mm V −2.1 mm from bregma, Paxinos mice atlas). The Hamilton syringe was connected to a micropump set at a flow-rate of 0.5 ml/min. Two minutes after the injection, the needle was removed and the surgical wound was sutured. The mice were returned to their cages and their conditions were monitored for 1 week. Experiments started at least 20 days post surgery.

### Immunocytochemistry

### In Cultured Cells:

Cell cultures were fixed in 4% paraformaldehyde in phosphate buffered saline (PBS), 0.12M in sucrose, and permeabilised with Triton X-100 (0.2%, Sigma). For F-actin detection, cells were stained with FITC (fluorescein isothiocyanate)-phalloidin (Sigma; working dilution 0.5 mg/ml in PBS) for 30 min at 37° C. Immunostaining was performed with the following primary antibodies: anti-microtubule-associated protein2 (MAP2), a marker of dendrites, anti-synaptophysin, a synaptic vesicle-associated protein, glial fibrillary acidic protein (GFAP), specifically identifying astrocytic cytoskeleton. All primary antibodies were purchased from Millipore, Mass., USA. After washing, samples were double-labelled with anti-mouse Alexa Fluor 488 and anti-rabbit 594 (Molecular Probes). Finally, after extensive washes, samples were mounted and observed with an Olympus BX51 fluorescence microscope or an Eclipse 80i Nikon Fluorescence Microscope, equipped with a VideoConfocal (ViCo) system.

### In Brain Tissues:

Mice were perfused with 4% paraformaldehyde in phosphate buffered saline (PBS), 0.12M in sucrose. The brains were removed, post-fixed for 30 min, washed in PBS, cryopreserved with increasing concentrations of sucrose in PBS and finally frozen in isopentane. Twenty-micron thick sections were cut at a cryostat and stored free-floating. Sections were immunolabelled for GFAP.

### Morphometric Analysis

### In Cultured Cells:

Morphometric analysis was conducted with the Optilab software (Graftek, Austin, Tex.). In MAP2-immunostained, pure hippocampal neurons at DIV 14, dendrite thickness was measured before the first dendritic branching. At least 60 dendrites were randomly chosen from two separate coverslips of the same culture, measured and averaged, to produce a single mean value for each culture. In hippocampal neurons co-cultured with astrocytes, after background subtraction, MAP2-positive area was measured as percentage of the total field area. Values obtained for each field (0.15 mm²) were pooled to obtain a single mean value for each neuronal culture. Statistical analyses were conducted by the nonparametric Wilcoxon test.

### Western Blot Analysis

### In Cultured Cells:

Cells were lysed in boiled sample buffer 1× (50 mM Tris-HCl, pH 6.8, 2% SDS, 10% glycerol, and 100 mM dithiothreitol). Twenty-five micrograms of total protein extracts were resolved by SDS-polyacrylamide gel electrophoresis (PAGE) and electrically transferred onto polyvinylidene difluoride membranes (Bio-Rad). Membranes were blocked with Tris-buffered saline-Tween 20 (TBS-T) (20 mM Tris-HCl, pH 7.4, 150 mM NaCl, and 0.02% Tween 20) containing 5% skimmed milk (Bio-Rad) for 30 min at room temperature, and then they were incubated overnight at 4° C. with primary antibodies diluted in TBS-T containing 2% milk. The following primary antibodies were used: mouse monoclonal anti-synaptophysin (Chemicon; 1:1000), rabbit polyclonal anti-spinophilin (Upstate; 1:1000), mouse monoclonal anti-SNAP-23 (Sy-Sy; 1:10000), rabbit polyclonal anti-GFAP (Millipore; 1:5000), mouse monoclonal anti-α-tubulin (Sigma; 1:10000). After extensive washing, immune complexes were detected with horseradish peroxidase-conjugated species-specific secondary antibodies (Jackson's) followed by enhanced chemiluminescence reaction (Amersham).

### Enzyme-Linked Immunosorbent Assay (ELISA)

### In Cultured Cells:

For detecting IL-1β and TNF-α, ILs ELISA kits were used following the manufacturer's instructions (BioVendor-Laboratorni, as.).

### In Brain Tissue:

For detecting IL-1β, hippocampal extract supernatants were prepared by dounce homogenisation and sonication in HEPES buffer containing a protease inhibitor mixture, followed by centrifugation as described (Craft et al., 2004). Levels of IL-1β in hippocampal supernatants were measured by ELISA (Biosource International) per the manufacturer's instructions.
For the determination of Aβ levels by ELISA, samples of mouse hippocampus were homogenised in ice-cold PBS containing 5M guanidine HCl and 13 proteases' inhibitor mixture (pH 8.0) (Calbiochem). Homogenates were mixed for 3-4 h at room temperature and centrifuged at 16,000×g for 20 min at 4° C. The supernatant was diluted 10-fold in Dulbecco's PBS (pH 7.4) containing 5% BSA and 0.03% Tween 20. Aβ1-42 levels in the diluted brain homogenates were quantified with a sandwich ELISA (BioSource International, Camarillo, Calif.) according to the manufacturer's instructions.
The content of cellular ATP was assayed luminometrically using the ATP lite Assay (Perkin Elmer-Cetus, Norwalk, Conn., USA), according to the procedure recommended by the manufacturer. In brief, tissues were homogenised in 50 ml of lysis buffer and mixed for 10 min. Forty microliters of substrate solution (Luciferase/Luciferin) was added to each sample. The luminescence was measured using a luminescence plate reader (Victor3-V, PerkinElmer Life Sciences). The ATP concentration was normalised to total tissue protein concentration estimated by Bradford protein assay (Bio-Rad).

### EEG and EEG Spectral Content

Surgery—DBA/2J male mice were used for the experiments. Chronic electrode implantation was performed after general anaesthesia with a mixture of ketamine (32 mg/kg) and xylazine (20 mg/kg) intraperitoneal, and local anaesthesia (xylocaine 2%, 0.1 mL subcutaneous). To record brain electric potentials (EEG), four epidural stainless steel electrodes were implanted (right and left anterior: 1.3 mm anterior to bregma and 1.5 mm lateral to sagittal suture, and right and left posterior: 3 mm posterior to bregma and 2.5 lateral to sagittal suture), the animals were allowed a recovery of 1 week before recording session began.

Data recording—The two traces, i.e. cortical and trigger were recorded on a STAC real-time analyser (Toenisvorst, Germany), after preamplification (1000×) of biologic signals, and all were over-sampled at 2.9 MHz, with 20 bit AID conversion, followed by re-sampling at 2.56 kHz, in order to avoid aliasing phenomena. Signals were recorded also on a GWI (Somerville, Mass., USA) system, set up by Analysa (Cuneo, Italy). EEG was recorded in free-moving mice for 20 min.

EEG and spectral analysis—Standard FFt analysis was performed on at least 20 min continuous EEG, devoid of movement artifacts, and mean spectral plots were built (±S.E.), from 1 to 512 Hz, with 2.5 Hz discrimination, for monitoring effects induced by drug on EEG spectral content.

### Results

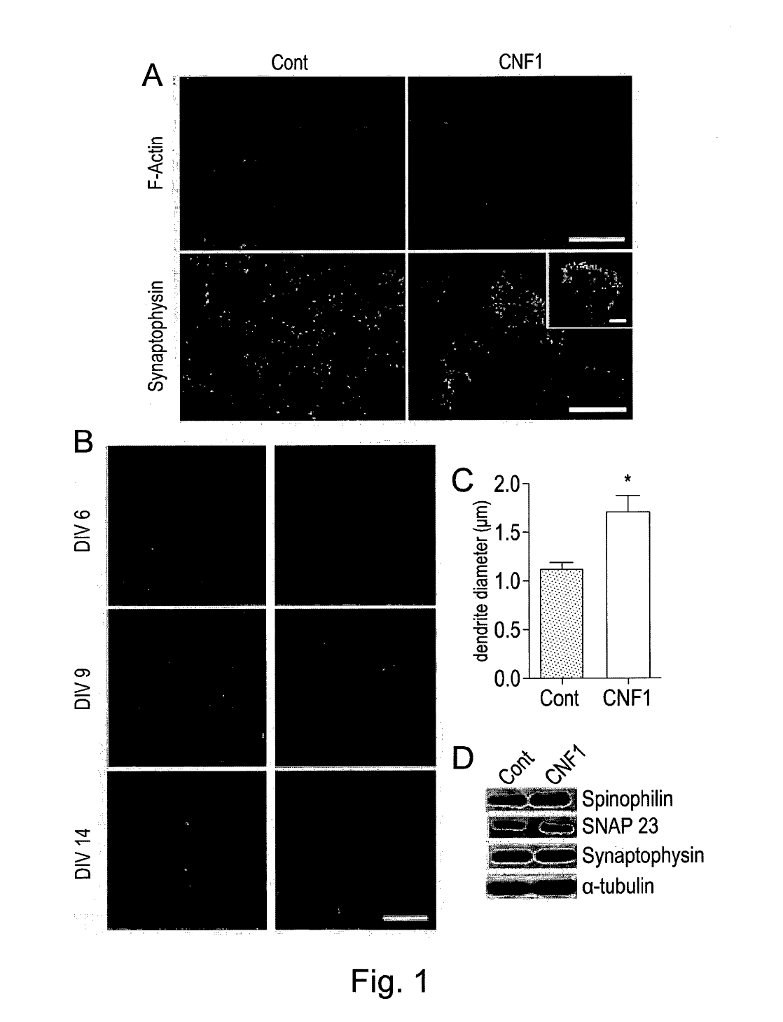

CNF1 modifies neuritic tree and synapse development in neurons during differentiation (FIG. 1).

Treatment of pure hippocampal neurons with CNF1 from DIV 2 profoundly affected neuronal differentiation. While in mature (DIV 14) control neurons actin-labelled neurites were long, thin and well defined, in CNF1-treated cultures, the neuritic tree and the cell bodies were covered with numerous and short protrusions, which gave the cells a spiny appearance (FIG. 1, panel A). CNF1-induced cytoskeletal changes were accompanied by a lack of synapse formation. In control cultures, at DIV 14, synaptophysin-positive synapses appeared as discrete dots, regularly distributed along the neurites. In contrast, in CNF1-treated hippocampal cultures, synaptophysin immunolabelling was dispersed and diffuse in the cell body and neurites, lacking the typical punctuated appearance (FIG. 1, panel A). It also clearly delineated growth cones, which were frequently observed in CNF1-treated cultures. Labelling of MAP2, a marker of the dendritic cytoskeleton, also highlighted CNF1-induced changes of the neuritic tree. In control pure hippocampal neurons, during differentiation, the MAP2-positive dendritic tree gradually enlarged and became ramified, with thin and smooth projections, until a complex network was formed (FIG. 1, panel B). Neuronal cell bodies maintained a round shape, with limited dimensions. When exposed to CNF1 from DIV 2 MAP-2-positive dendrites appeared thicker and more tortuous. Thin ramifications were lacking. Neuronal cell bodies were large, with a veil-like appearance (FIG. 1, panel B). Morphometric analysis confirmed that the thickness of the dendrites was increased in cells challenged with the toxin (FIG. 1, panel C, *=p<0.05, Wilcoxon Matched Pairs test).

Western blot analysis showed that the levels of synaptic proteins, such as synaptophysin and SNAP23, or of components of the dendritic tree, such as spinophilin, were similar in CNF1-treated and control cultures (FIG. 1, panel D).

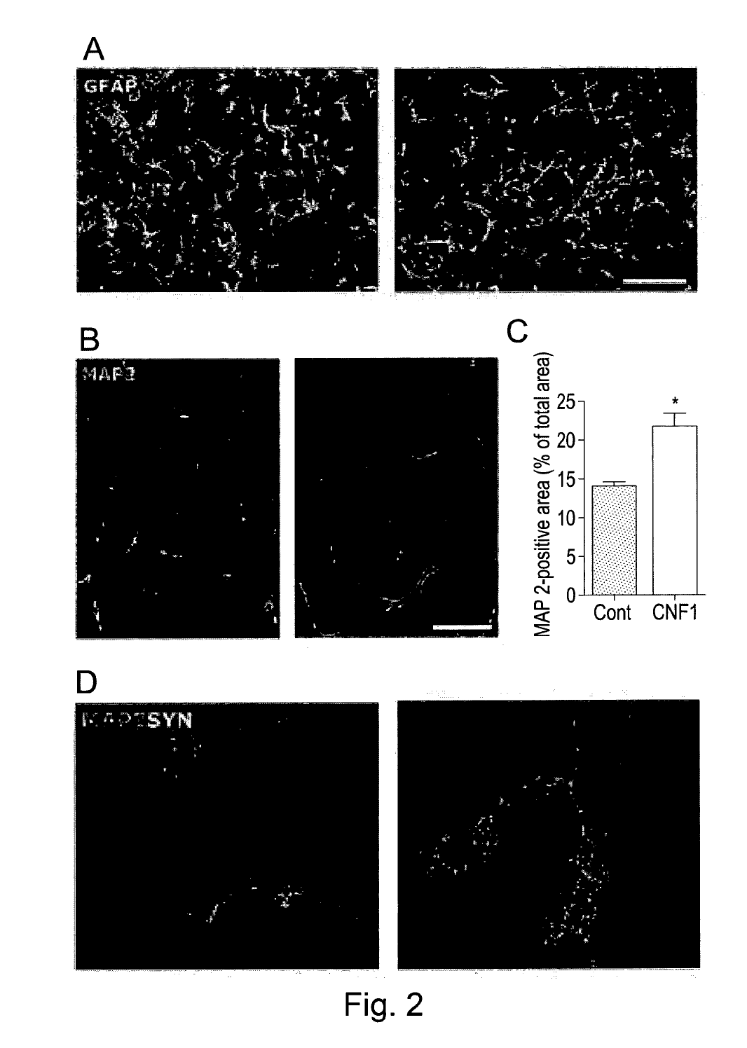

CNF1-treated astrocytes provide a more efficient substrate to neuritogenesis and synaptogenesis (FIG. 2).

In the protocols so far described, CNF1 was administered directly to neurons, in the presence or absence of astrocytes. However, it seemed possible that in in vivo treatment, where CNF1 is delivered by means of intracerebroventricular injections, the toxin first interacts with ependymal cells, which line the ventricles, and then with astroglial cells, which surround the ependymal layer. Accordingly, we decided to find out whether the beneficial effects observed in vivo could be mediated by the interaction of CNF1 with astrocytes. To address this question, we treated pure astrocytic cell cultures with CNF1 and analysed how this treatment affected astrocytic ability to support neuronal cell growth. At a difference from the experiments conducted in the art, hippocampal neurons, growing on CNF1-treated astrocytes (FIG. 2, panel A), but in absence of direct CNF1 influence, produced a much more abundant dendritic tree, with richer branching, creating a confluent network, as shown by MAP-2 immunolabelling (FIG. 2, panel B). Morphometric analysis confirmed the augmented MAP-2-positive area (FIG. 2, panel C, *=p<0.05, Wilcoxon Matched Pairs test). Furthermore, the enlargement of the dendritic tree was accompanied by an increased formation of synapses, as shown by synaptophysin immunolabelling (FIG. 2, panel D).

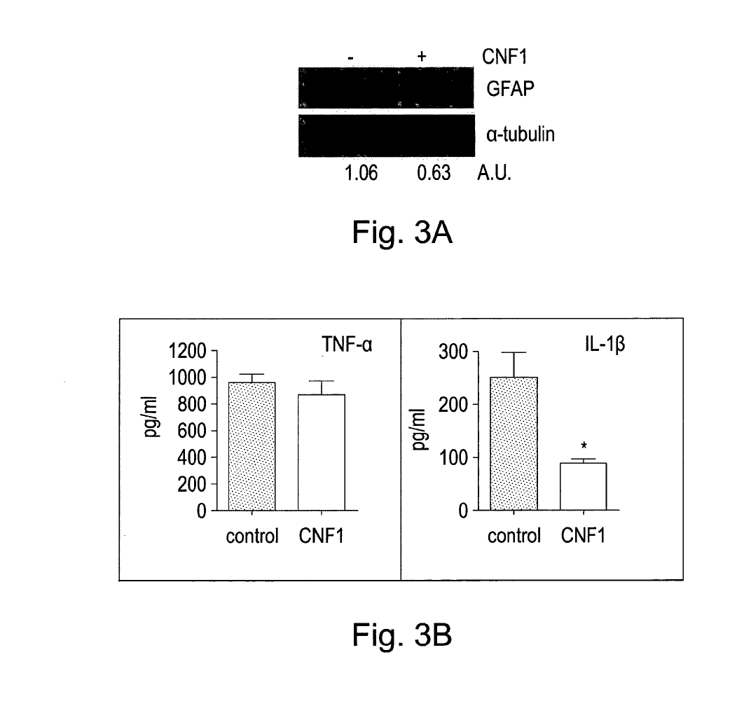

CNF1 treatment endows astrocytes with a neuroprotective phenotype (FIG. 3).

In neuronal/astrocytic co-cultures, GFAP immunolabelling was less evident in CNF1 treated astrocytes than in control cultures (FIG. 3, panel A). Since a reduction in GFAP content has been put in relation to increased astrocytic-induced dendritogenesis (Middeldorp and Hol 2011), to confirm this finding, we grew pure astrocytic cultures and analysed GFAP content by Western blotting. GFAP was evidently reduced after CNF1 treatment. Furthermore, in the same cultures, we measured the expression of TNF-α and IL-1β after challenge with CNF1. We found that, whereas the expression of the pro-inflammatory cytokine TNF-α was unaffected, IL-1β was significantly decreased in astrocytes challenged with the toxin (FIG. 3, panel B, *=p<0.05, t-Student test). Since IL-1β directly impairs neurogenesis (Kuzumaki et al., 2010), its decrease is consistent with the observed positive modulation of dendritic growth after treatment with CNF1.

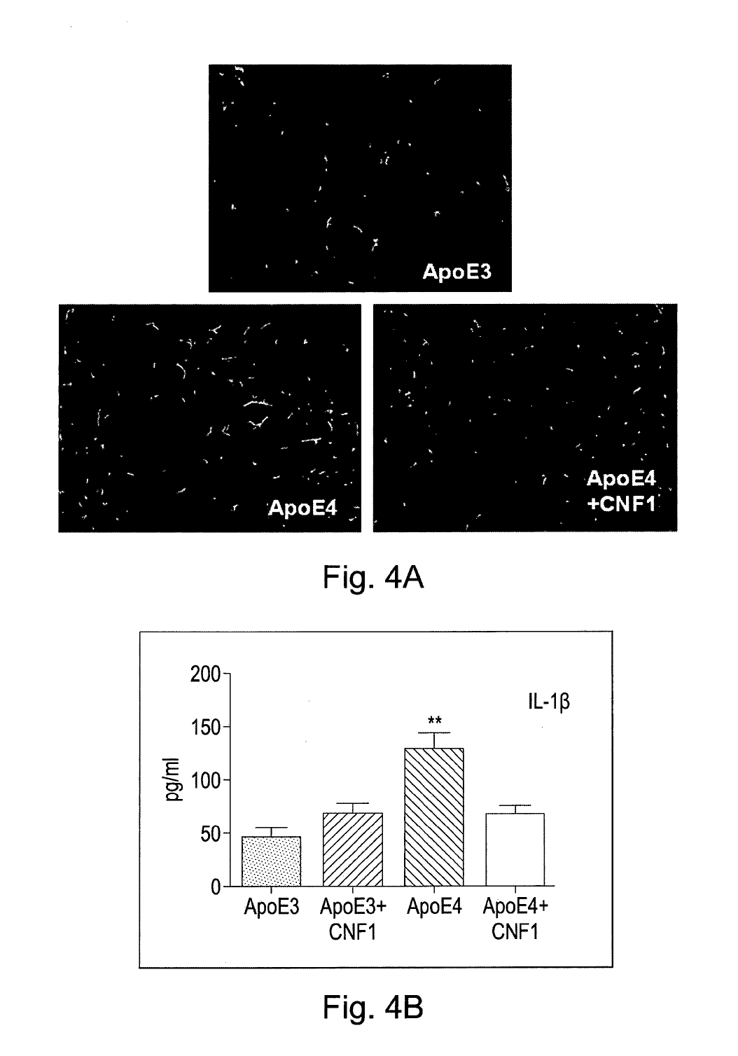

Astrogliosis and neuroinflammation in ApoE4 mice is reversed by CNF1 (FIG. 4).

An immunohistochemical analysis was conducted to characterise astrocytic components in frozen brain sections from ApoE4 and ApoE3 mice (FIG. 4, panel A). Immunoreactivity of GFAP, a specific marker of astrocytic cytoskeleton, was analysed in the hippocampus of ApoE4 mice where astrocytes showed features of astrogliosis, with thickened and branched ramifications, when compared to ApoE3 animals. CNF1 reverted the gliotic phenotypes, reducing GFAP expression (FIG. 4, panel A).

Given the important physiological role played by cytokines in synaptic plasticity, neurogenesis, and neuromodulation, we evaluated whether their expression is altered in ApoE4 mice and CNF1 activity can influence their expression in mouse brain tissue. IL-1β levels were increased but not significantly in ApoE3 mice whereas the overexpression of the cytokine in ApoE4 animals was significantly reverted by CNF1 (FIG. 4, panel B, *=p<0.05, t-Student test).

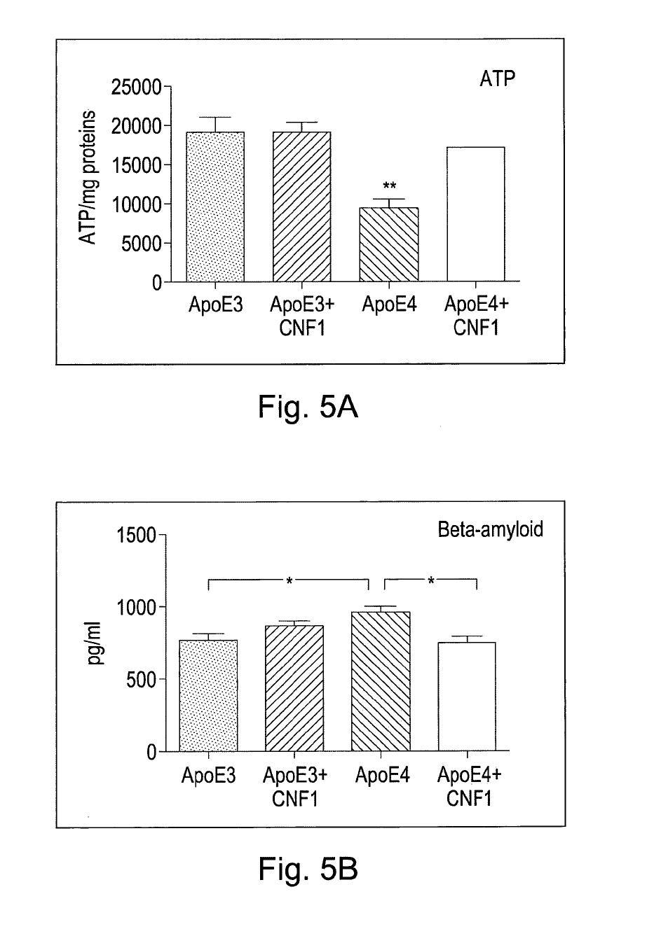

CNF1 increases the ATP levels and decrease the β amyloid expression in ApoE4 mice (FIG. 5)

Astrocytes, probably the most widespread source of ATP release in the CNS, have a number of mechanisms for the release of ATP, which can be considered to be a ‘gliotransmitter’. In hippocampus of ApoE4 mice, ATP levels were significantly reduced with respect to those detected in ApoE3. CNF1 treatment caused no changes in ApoE3 control mice while it completely restored the ATP levels in ApoE4 (FIG. 5, panel A, *=p<0.05, t-Student test).

In the hippocampus of ApoE4 mice the expression of β amyloid was higher than that of control ApoE3 (FIG. 5, panel B, *=p<0.05, t-Student test). In ApoE4 hippocampus, CNF1 diminished β amyloid expression to control levels.

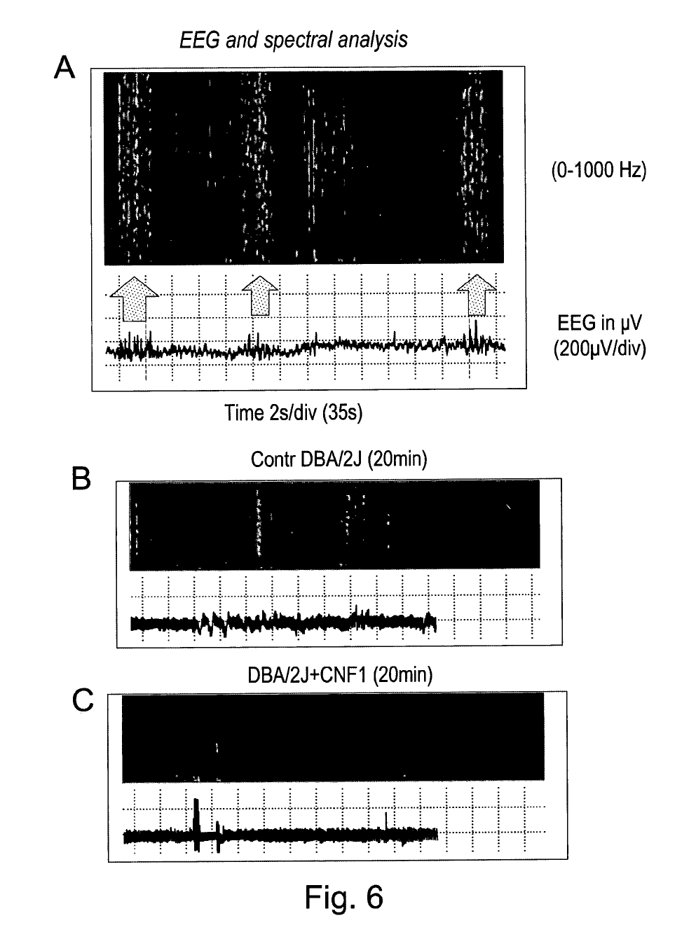

CNF1 counteracts spontaneous seizure in DBA/2J (D2) mice (FIG. 6)

As described above, reactive astrogliosis is involved also in all forms of seizures. For example, spontaneous seizure is characterised by both neuronal and astroglial dysfunction and inflammatory changes, such as an increase in GFAP-immunoreactivity and pro-inflammatory cytokine levels (Gahring et al., 1997; Li et al., 2007; Pernot et al., 2011). Thus, we examined electroencephalogram (EEG) and the evaluation in frequency and time domain in the somatosensitive cortex of a genetic spontaneous seizures model, the DBA/2J (D2) mouse where the EEG spontaneous seizures recorded in the low part of the figure correspond to high-frequency bursts elaborated as shaded lines in the upper part with a tonal range scaling (FIG. 6, panel A). Our preliminary data show a different result in 20 minutes Time/frequency EEG analysis recorded in controls (FIG. 6, panel B) versus mice treated with a single i.c.v. injection of CNF1, which is able to significantly counteract the low frequency (7-8 cycles per second) spike and wave complexes, that characterise this animal model (FIG. 6, panel C).

### REFERENCES

Bortolato M, Frau R, Orrù M, Fà M, Dessi C, Puligheddu M, Barberini L, Pillolla G, Polizzi L, Santoni F, Mereu G, Marrosu F (2010) GABAB receptor activation exacerbates spontaneous spike-and-wave discharges in DBA/2J mice. Seizure. 19(4):226-31.
Cerri C, Fabbri A, Vannini E, Spolidoro M, Costa M, Maffei L, Fiorentini C and Caleo M (2011) Activation of Rho GTPases triggers structural remodeling and functional plasticity in the adult rat visual cortex. J Neurosci, 31(42):15163-72.
Chen and Yan X S D, Mitochondrial Aβ: a potential cause of metabolic dysfunction in Alzheimer's disease. IUBMB Life. 2006 December; 58 (12) :686-94,
Chen C J, Liao S L, Kuo J S (2000) Gliotoxic action of glutamate on cultured astrocytes. J Neurochem 75(4): 1557-1565
Craft J M et al., (2004) Aminopyridazines inhibit β-amyloid-induced glial activation and neuronal damage in vivo. Neurobiol Aging 25: 1283-1292
De Viti S, Martino A, Musilli M, Fiorentini C, Diana G (2010) The rho GTPase activating CNF1 improves associative working memory for object-in-place. Behav Brain Res 212(1): 78-83.
Diana G, Valentini G, Travaglione S, Falzano L, Pieri M, et al. (2007) Enhancement of learning and memory after activation of cerebral rho GTPases. Proc Natl Acad Sci USA 104(2): 636-641.
Dumanis S B, Tesoriero J A, Babus L W, Nguyen M T, Trotter J H, Ladu M J, Weeber E J, Turner R S, Xu B, Rebeck G W, Hoe H S. (2009) ApoE4 decreases spine density and dendritic complexity in cortical neurons in vivo. J Neurosci. 29(48):15317-22.
Emirandetti A, Graciele Zanon R, Sabha M, Jr, de Oliveira A L (2006) Astrocyte reactivity influences the number of presynaptic terminals apposed to spinal motoneurons after axotomy. Brain Res 1095(1): 35-42.
Falzano L, Fiorentini C, Donelli G, Michel E, Kocks C, Cossart P, Cabanié L, Oswald E, Boquet P. Induction of phagocytic behaviour in human epithelial cells by Escherichia coli cytotoxic necrotizing factor type 1. Mol Microbiol 9, 1247-1254 (1993).
Flatau G, Lemichez E, Gauthier M, Chardin P, Paris S, et al. (1997) Toxin-induced activation of the G protein p21 rho by deamidation of glutamine. Nature 387(6634): 729-733.
Gahring L C, White H S, Skradski S L, Carlson N G, Rogers S W. Interleukin-1α in the brain is induced by audiogenic seizure. Neurobiol Dis. 1997; 3(4):263-9.
Galietta G, Giuliani G, Loizzo A, Amat AG, Fumagalli E, De Feo V, Quaranta E, Paladino L, Capasso A (2005) Neurophysiological studies of Heteropteris glabra Hak. & Am. (Malpighiaceae) in DBA/2J mice J Ethnopharmacol. 21; 97(3):415-9.
Garay P A, McAllister A K (2010) Novel roles for immune molecules in neural development: Implications for neurodevelopmental disorders. Front Synaptic Neurosci 2: 136.
Gilmore J H, Fredrik Jarskog L, Vadlamudi S, Lauder J M (2004) Prenatal infection and risk for schizophrenia: IL-1β, IL-6, and TNFα inhibit cortical neuron dendrite development. Neuropsychopharmacology 29(7): 1221-1229.
Glass C K, Saijo K, Winner B, Marchetto M C, Gage F H. (2010) Mechanisms underlying inflammation in neurodegeneration. Cell 140(6): 918-934.
Hotulainen P, Hoogenraad C C (2010) Actin in dendritic spines: Connecting dynamics to function. J Cell Biol 189(4): 619-629.
Kuzumaki N, Ikegami D, Imai S, Narita M, Tamura R, et al. (2010) Enhanced IL-1β production in response to the activation of hippocampal glial cells impairs neurogenesis in aged mice. Synapse 64(9): 721-728.
Li T, Quan Lan J, Fredholm B B, Simon R P, Boison D. Neuron Glia Biol. Adenosine dysfunction in astrogliosis: cause for seizure generation? 2007 November; 3(4):353-66.
Malchiodi-Albedi F, Domenici M R, Paradisi S, Bernardo A, Ajmone-Cat M A, et al. (2001) Astrocytes contribute to neuronal impairment in β A toxicity increasing apoptosis in rat hippocampal neurons. Glia 34 (1): 68-72.
Menet V, Gimenez y Ribotta M, Chauvet N, Drian M J, Lannoy J, et al. (2001) Inactivation of the glial fibrillary acidic protein gene, but not that of vimentin, improves neuronal survival and neurite growth by modifying adhesion molecule expression. J Neurosci 21 (16): 6147-6158,
Middeldorp J, Hol E M (2011) GFAP in health and disease. Prog Neurobiol 93(3): 421-443.
Pavone F, Luvisetto S, Marinelli S, Straface E, Fabbri A, et al. (2009) The rac GTPase-activating bacterial protein toxin CNF1 induces analgesia up-regulating mu-opioid receptors. Pain 145 (1-2): 219-229.
Pernot F, Heinrich C, Barbier L, Peinnequin A, Carpentier P, Dhote F, Baille V, Beaup C, Depaulis A, Dorandeu F. Inflammatory changes during epileptogenesis and spontaneous seizures in a mouse model of mesiotemporal lobe epilepsy. Epilepsia. 2011 Sep. 28. doi: 10.1111/j.1528-1167.2011.03273.x. [Epub ahead of print]
Reid C A, Kim T H, Berkovic S F, Petrou S (2011) Low blood glucose precipitates spike-and-wave activity in genetically predisposed animals. Epilepsia. 52(1):115-20
Rozovsky I, Wei M, Morgan T E, Finch C E (2005) Reversible age impairments in neurite outgrowth by manipulations of astrocytic GFAP. Neurobiol Aging 26 (5): 705-715
Schmidt G, Sehr P, Wilm M, Selzer J, Mann M (1997) Gln 63 of rho is deamidated by Escherichia coli cytotoxic necrotizing factor-1. Nature 387(6634): 725-729.
Sofroniew M V and Vinters H V (2010), Astrocytes: biology and pathology. Acta Neuropathol 119: 7-35.
Tan H O, Reid C A, Chiu C, Jones M V, Petrou S (2008) Increased thalamic inhibition in the absence seizure prone DBA/2J mouse. Epilepsia. May; 49(5):921-5.
Tong L, Balazs R, Soiampornkul R, Thangnipon W, Cotman C W (2008) Interleukin-1 β impairs brain derived neurotrophic factor-induced signal transduction. Neurobiol Aging 29 (9): 1380-1393.
Verghese P B, Castellano J M, Holtzman D M (2011) Apolipoprotein E in Alzheimer's disease and other neurological disorders. Lancet Neurol. 10(3):241-52.
Vincent B, Smith J D. Astrocytes down-regulate neuronal β-amyloid precursor protein expression and modify its processing in an apolipoprotein E isoform-specific manner. Eur J Neurosci. 2001 July; 14(2):256-66)
Whitney N P, Eidem T M, Peng H, Huang Y, Zheng J C (2009) Inflammation mediates varying effects in neurogenesis: Relevance to the pathogenesis of brain injury and neurodegenerative disorders. J Neurochem 108 (6): 1343-1359.

### Example 2

### Effects of CNF1 on Rat Primary Astrocyte Cultures

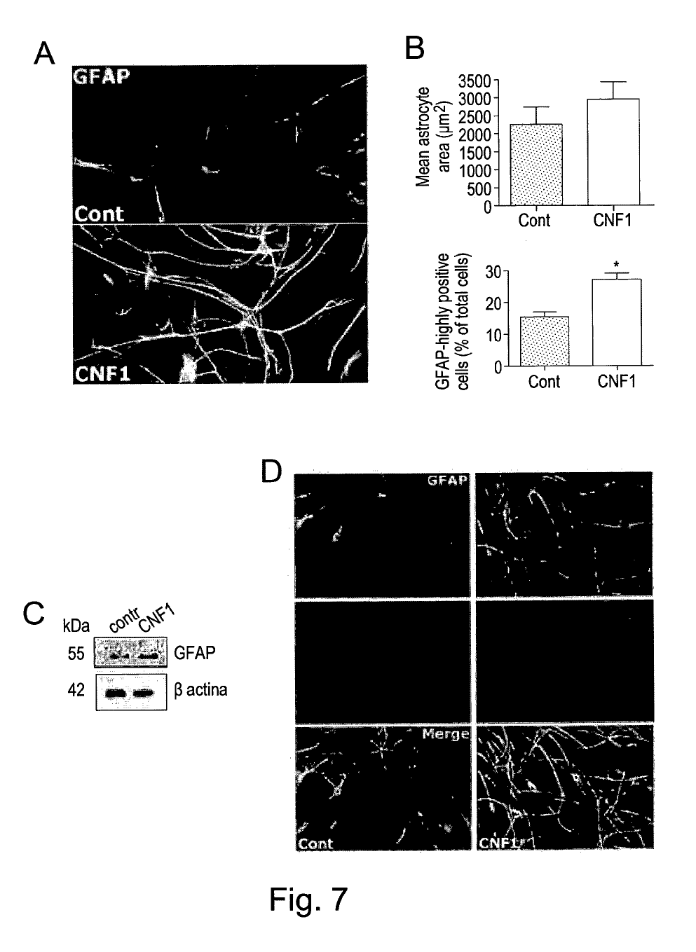

Cultured astrocytes exhibit a flat/epitelioid phenotype much different from the star-like phenotype of tissue astrocytes. Upon exposure to treatments that affect the Rho GTPases, however, flat astrocytes undergo stellation, with restructuring of cytoskeleton and outgrowth of processes with lamellipodia, assuming a phenotype closer to that exhibited in situ. This is shown in FIG. 7, which shows that treatment with CNF1 for 48 h induces in primary cultured cortical astrocytes an increase in the growth of thin and ramified projections in astrocytes and in GFAP reactivity (A), documented by morphometric analysis (B) and western blotting (C), reminiscent of the phenomenon termed “astrocyte stellation”. Increase in ramifications is also evident in vimentin immunolabeled astrocytes (D).

### Effects of CNF1 on EEG of an Absence Seizures Mouse Model

Astrocytic dysfunctions are known to be present in absence seizures and, in general, in epilepsy (Melø et al., 2007; Sofroniew and Vinters, 2010; Akin et al., 2011; Pirttimaki et al., 2012; Onat et al., 2012). In this report, we studied CNF1 effects in an inbred strain of mice, the DBA/2J, showing an age-dependent form of epilepsy very similar to the absence seizures in humans (Capasso et al, 1994a and 1994b). We analyzed the fast neuronal network oscillations in the gamma range (30-90 Hz and over) that have been implicated in some forms of seizures (spikes, poli-spikes, spike-and-dome sequences). All these phenomena were accompanied by strong increase of fast and very fast nervous electrical oscillations, similar to those described by Kann's group (Kann et al 2005; Kann et al, 2011), and this suggested us to evaluate the epileptiform seizures in mouse models through the analysis of fast and very fast frequencies recorded in the brain of mice.

We have found that the fast neuronal oscillations can be monitored through a new approach in the electroencephalographic (EEG) analysis of seizures, and we decided to study whether the use of, CNF1 could modulate epileptiform seizures of DBA/2J mice for longer times, and whether this could be monitored through an EEG analysis of high frequencies in the mouse.

### Materials and Methods

Ethical Guidelines. All procedures were carried out in accordance with the guidelines of the Council of European Communities and the approval of Bioethical Committee of the Italian National Institutes of Health. All mice were housed in a central facility and maintained under controlled conditions of normal humidity and temperature, with standard alternating 12-h periods of light and darkness. Animals had free access to water and food. Mucedola S.r.l. (Settimo Milanese, Italy) supplied the diet, which contained 3.95 kcal/g equivalent to assimilable 2.7 kcal/g.

Methods and Materials. Experimental animals were male inbred DBA/2J mice, aged 16-20 weeks, which were purchased from Charles River Italia (Calco-Lecco, Italy). At least 8 days after arrival, mice received general (xylazine-ketamine) and local (lidocaine) anaesthesia, and were inserted appropriately in a stereotactic apparatus. Then, mice were implanted with chronic cortical stainless steel electrodes on the right frontal area, and on the right and left sensorimotor areas, according to previously described techniques (Lopez et al, 2002; Loizzo et al, 2012). During the same surgery approach, a hole was drilled in the left frontal area, and a needle connected to a microsyringe was inserted through the brain cortex down to the left cerebral ventricle. Through the syringe, 3 microliters of sterile saline solution were injected into ten animals (control mice, saline). In random sequence, in ten more animals were injected 3 microliters of a 10⁻¹⁰M CNF1 solution (treated mice, CNF1).

Recording of cerebral electrical activity. At least eight days after surgery, mice underwent recording of the cerebral electrical activity in steady-state conditions (EEG), in a sound- light- and electrically-shielded room, always in the same hours of the day (10:00-13:00), according to previously published procedures, with some modifications (Capasso & Loizzo, 2003; Capasso et al, 2003). Briefly, mice electrodes were connected to a digital amplifier-recording system, set up in our laboratories with the technical assistance of Analysa (Cuneo, Italy). Signals from the two derivations of the right hemisphere were amplified (1000×), band-pass filtered (1 to 500 Hz) sampled at 2.5 kHz and recorded on disk in periodograms of 1200 s. In each mouse 4 to 8 periodograms were recorded, i.e., up a total of 80-160 minutes.

EEG elaboration. EEG spectrograms were elaborated according to a protocol of time- and frequency-domain analysis, according to parameters set up and published from our laboratories (Galietta et al, 2005; Vyssotski et al, 2009). Briefly, spectrograms were recalled on the display, and were elaborated according to the Soundscope protocol, in blocks of 600 s. The protocol shows the power-spectral analysis of the entire 600 s block through parallel vertical lines, which represent the power of spectral bands in pseudocolor, where the higher frequencies of the bands are expressed in dark colors (lower power bands) or in brilliant colors (yellow-red) which correspond to higher spectral power, from 1 to 500 Hz, sometimes up to 1kHz. Analysis parameters are set according to the specific target for each investigation, including the physiologic states of the animal. In the present investigation the spectral plot was set at 1 to 0.5 kHz, in lines computed on 256 consecutive points of the original tracing. Thus, when EEG blocks represent preponderance of low frequencies, the lines give an appearance of black display, with some scattered colored lines. When high frequencies are better represented in the EEG, colored lines grow more and more, up to a maximal expression over the whole display covered with red-yellow lines everywhere.

Data are gathered through scores from 0 to 6 attributed to various spectrograms. Score 0 effectively shows black, while score 6, full spectrogram corresponding to 600 sec, is filled with high frequencies showing a lot of colour.

### Results and Conclusions

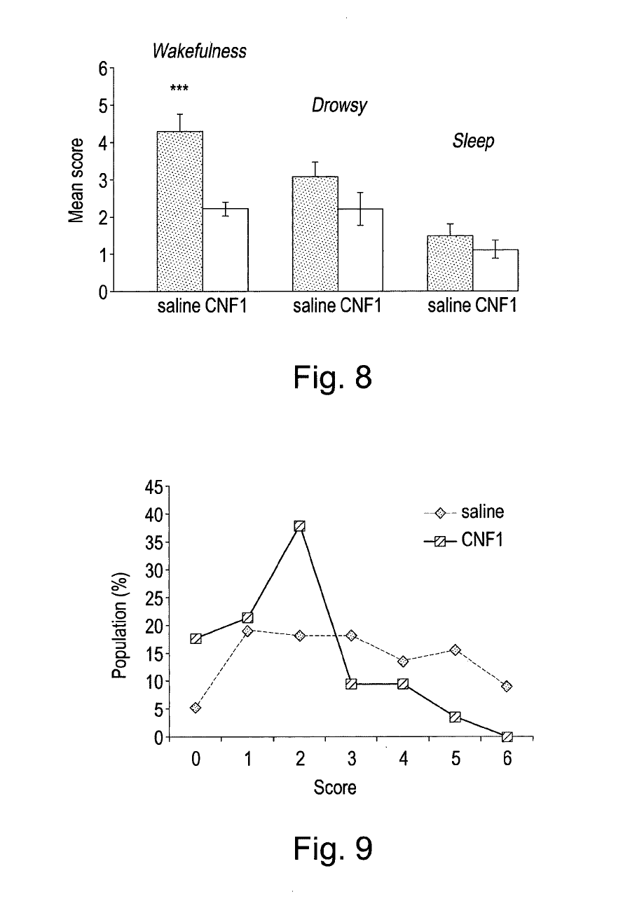

Spectrograms computed from EEG of absent seizures model (DBA/2J) treated with CNF1, showed a significant decrement of high frequencies episodes versus controls in wakefulness physiological state. A difference was also recorded during drowsy and sleep periods, but was not statistically significant (FIG. 8). FIG. 9 shows distribution of scores in percent of total for control and CNF1 treated animals. Lower scores are prevalent in CNF1 treated animals (0 to 2); while high scores where much more evident in saline treated animals (score 3 to 6) (FIG. 8).

In FIG. 8, the ordinate shows the mean score attributed to periods of 600 sec/EEG (High frequency score) in control and treated DBA/2J mice. Statistical analysis non parametric t-test, P=0.0002 (p<0.001 ***) (n=10 per group).

FIG. 9 shows the distribution of scores for the two populations (saline and CNF1 treated). Physiological states are cumulative.

It can be seen that single intracerebroventricular treatment of DBA/2J mice with even very small doses of CNF1 is able to consistently attenuate high and very high frequencies in the EEG of DBA/2J mice, and consequently to strongly attenuate epileptiform spikes, polispikes, and spike-and-dome complex phenomena.

### REFERENCES

Akin D, Ravizza T, Maroso M, Carcak N, Eryigit T, Vanzulli I, Aker R G, Vezzani A, Onat FY. IL-1β is induced in reactive astrocytes in the somatosensory cortex of rats with genetic absence epilepsy at the onset of spike-and-wave discharges, and contributes to their occurrence. Neurobiol Dis. 2011 December; 44(3):259-69.
Capasso A, Bianchi A, Loizzo A. Nitric oxide is involved in the expression of neocortical spike-and-wave spindling episodes in DBA/2J mice. J Pharm Pharmacol. 2003 August; 55(8):1115-9.
Capasso A, Loizzo A. Alpha2-adrenoceptor antagonist SL 84.0418 reduces the expression of neocortical spike-and-wave spindling episodes in DBA/2J mice. Prog Neuropsychopharmacol Biol Psychiatry. 2003 February; 27(1):141-5.
Capasso A, Sorrentino L, Di Giannuario A, Palazzesi S, Pieretti S, Loizzo A. Dexamethasone and hormones related to the hypothalamic-pituitary-adrenal axis modulate inherited neocortical spindling in DBA/2J mice. Neuropsychobiology. 1994b; 29(3):143-51.
Capasso A, Sorrentino L, Di Giannuario A, Pieretti S, Loizzo A. Effect of des-tyrosine-gamma-endorphin on neocortical spike-and-wave spindling in DBA/2J mice. Eur J Pharmacol. 1994a: 11; 261(1-2):209-12.
Galietta G, Giuliani G, Loizzo A, Amat A G, Fumagalli E, De Feo V, Quaranta E, Paladino L, Capasso A. Neurophysiological studies of Heteropteris glabra Hok. & Am. (Malpighiaceae) in DBA/2J mice. J Ethnopharmacol. 2005 Mar. 21; 97(3):415-9.
Kann O, Huchzermeyer C, Kovács R, Wirtz S, Schuelke M. Gamma oscillations in the hippocampus require high complex I gene expression and strong functional performance of mitochondria. Brain. 2011 February; 134(Pt 2):345-58.
Kann O, Kovács R, Njunting M, Behrens C J, Otáhal J, Lehmann T N, Gabriel S, Heinemann U. Metabolic dysfunction during neuronal activation in the ex vivo hippocampus from chronic epileptic rats and humans. Brain. 2005 October; 128(Pt 10):2396-407.
Loizzo A, Spampinato S M, Campana G, Vella S, Fortuna A, Costa L, Capasso A, Monteleone P, Renzi P, Loizzo S. Enhanced brain performance in mice following postnatal stress. J Endocrinol. 2012 December; 215(3):413-24.
Lopez L, Brusa A, Fadda A, Loizzo S, Martinangeli A, Sannita W G, Loizzo A. Modulation of flash stimulation intensity and frequency: effects on visual evoked potentials and oscillatory potentials recorded in awake, freely moving mice. Behav Brain Res. 2002 Apr. 1; 131(1-2):105-14.
Melø T M, Sonnewald U, Bastholm I A, Nehlig A. Astrocytes may play a role in the etiology of absence epilepsy: a comparison between immature GAERS not yet expressing seizures and adults. Neurobiol Dis. 2007 November; 28(2):227-35.
Onat F. Astrocytes and absence epilepsy. Br J Pharmacol. 2012 Nov. 12 doi: 10.1111/bph.12050. [Epub ahead of print]
Pirttimaki T M, Parri H R, Crunelli V. Astrocytic GAT-1 dysfunction in experimental absence seizures. J Physiol. 2012 Nov. 19.
Racchetti G, D'Alessandro R, Meldolesi J. Astrocyte stellation, a process dependent on Rac1 is sustained by the regulated exocytosis of enlargeosomes. Glia. 2012 March; 60(3):465-75.
Sofroniew M V, Vinters H V. Astrocytes: biology and pathology. Acta Neuropathol. 2010 January; 119(1):7-35.
Vyssotski A L, Dell'Omo G, Dell'Ariccia G, Abramchuk A N, Serkov A N, Latanov A V, Loizzo A, Wolfer D P, Lipp H P. EEG responses to visual landmarks in flying pigeons. Curr Biol. 2009 Jul. 28; 19(14):1159-66.

```
A preferred naturally occurring CNF1 sequence
is SEQ ID NO 1
    1
MGNQWQQKYL LEYNELVSNF PSPERVVSDY IKNCFKTDLP
WFSRIDPDNA YFICFSQNRS
  61
NSRSYTGWDH LGKYKTEVLT LTQAALINIG YRFDVFDDAN
SRTGIYKTKS ADVFNEENEE
121
KMLPSEYLHL LQKCDFAGVY GKTLSDYWSK YYDKFKLLLK
NYYISSALYL YKNGELDERE
181
YNFSMNALNR SDNISLLFFD IYGYYASDIF VAKNNDKVML
FIPGAKKPFL FKKNIADLRL
241
TLKELIKDSD NKQLLSQHFS LYSRQDGVSY AGVNSVLHAI
ENDGNFNESY FLYSNKTLSN
301
KDVFDAIAIS VKKRSFSDGD IVIKSNSEAQ RDYALTILQT
ILSMTPIFDI VVPEVSVPLG
361
LGIITSSMGI SFDQLINGDT YEERRSAIPG LATNAVLLGL
SFAIPLLISK AGINQEVLSS
421
VINNEGRTLN ETNIDIFLKE YGIAEDSISS TNLLDVKLKS
SGQHVNIVKL SDEDNQIVAV
481
KGSSLSGIYY EVDIETGYEI LSRRTYRTEY NNEILWTRGG
GLKGGQPFDF ESLNIPVFFK
541
DEPYSAVTGS PLSFINDDSS LLYPDTNPKL PQPTSEMDIV
NYVKGSGSFG DRFVTLMRGA
601
TEEEAWNIAS YHTAGGSTEE LHEILLGQGP QSSLGFTEYT
SNVNSADAAS RRHFLVVIKV
661
HVKYITNNNV SYVNHWAIPD EAPVEVLAVV DRRFNFPEPS
TPPDISTIRK LLSLRYFKES
721
IESTSKSNFQ KLSRGNIDVL KGRGSISSTR QRAIYPYFEA
ANADEQQPLF FYIKKDRFDN
781
HGYDQYFYDN TVGLNGIPTL NTYTGEIPSD SSSLGSTYWK
KYNLTNETSI IRVSNSARGA
841
NGIKIALEEV QEGKPVIITS GNLSGCTTIV ARKEGYIYKV
HTGTTKSLAG FTSTTGVKKA
901
VEVLELLTKE PIPRVEGIMS NDFLVDYLSE NFEDSLITYS
SSEKKPDSQI TIIRDNVSVF
961
PYFLDNIPEH GFGTSATVLV RVDGNVVVRS LSESYSLNAD
ASEISVLKVF SKKF
```

1. CNF1 for use in the treatment of a condition associated with astrocyte dysfunction.

2. CNF1 for use according to claim 1, wherein said condition is associated with neuroinflammation and/or astrogliosis.

3. CNF1 for use in restoring dysfunctional astrocytes to normal functioning.

4. CNF1 for use according to any preceding claim, wherein the treatment is of a patient, and the patient is human.

5. CNF1 for use according to any preceding claim, wherein said CNF1 is provided in a form suitable for administration by injection or drip.

6. CNF1 for use according to claim 1, which has the sequence of SEQ ID NO. 1, or is a mutant or variant thereof capable of activating Rho GTPases.

7. CNF1 for use according to any preceding claim, which has the sequence of SEQ ID NO. 1.

8. CNF1 for use according to any preceding claim, wherein the condition is diabetic retinopathy.

9. CNF1 for use according to any of claims 1 to 7, wherein the condition is a seizure disorder

10. CNF1 for use according to any of claims 1 to 7, wherein the condition is epilepsy.

11. CNF1 for use according to any of claims 1 to 7, wherein the condition is glaucoma.

12. CNF1 for use according to any of claims 1 to 7, wherein the condition is Parkinson's disease.

13. CNF1 for use according to any of claims 1 to 7, wherein the condition is tauopathic.

14. CNF1 for use according to any of claims 1 to 7, wherein the condition is cerebral amyloid angiopathy.

15. CNF1 for use according to any of claims 1 to 7, wherein the condition is Alzheimer's disease.


SEQUENCE LISTING (1 sequence)

SEQ ID NO: 1 - protein, 1014 aa, Escherichia coli
  Escherichia coli strain 392 ISS cytotoxic necrotizing factor 1 (CNF1)
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSRTGIYKTKSADVFNEENEEKMLPSEYLHLLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDNKQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF